(19) United States
(12) Patent Application Publication
(10) Pub. No.: US 2019/0313632 A1
(43) Pub. Date: Oct. 17, 2019

(54) CRYOPRESERVATION MEDIUM AND METHOD TO PREVENT RECRYSTALLIZATION

(71) Applicants: XU HAN, COLUMBIA, MO (US); YE YUAN, HIGHLANDS RANCH, CO (US); ROBERT MICHAEL ROBERTS, COLUMBIA, MO (US)

(72) Inventors: XU HAN, COLUMBIA, MO (US); YE YUAN, HIGHLANDS RANCH, CO (US); ROBERT MICHAEL ROBERTS, COLUMBIA, MO (US)

(21) Appl. No.: 16/300,970

(22) PCT Filed: May 15, 2017
(86) PCT No.: PCT/US17/32606

§ 371 Date: Nov. 13, 2018

Related U.S. Application Data
(60) Provisional application No. 62/336,142, filed on May 13, 2016.

(51) Int. Cl. A01N 1/02; C12N 1/04

(52) U.S. Cl. CPC A01N 1/0231; C08L 71/03; A01N 1/0221; C12N 1/04

(57) ABSTRACT

The present invention is directed to a medium for preserving cells at non-cryogenic freezing temperatures. The medium comprises a hydrophilic and nontoxic polymer or other macromolecule, an aqueous liquid, and a cryoprotectant. The molecules of the macromolecule form compact three-dimensional structures that are spherical in shape when dissolved in the aqueous liquid. The medium of the present invention can be used for long-term storage of cells at non-cryogenic temperatures with outcomes similar to those seen with storage at cryogenic temperatures.

### CROSS-REFERENCE TO RELATED APPLICATIONS

This application is based on and claims priority to U.S. Provisional Application Ser. No. 62/336,142 filed on May 13, 2016, which is hereby incorporated herein by reference.

### BACKGROUND OF THE INVENTION

### 1. Field of the Invention

The present invention is directed to the fields of cryobiology and cryopreservation.

### 2. Description of Related Art

Current long term storage of cell stocks routinely requires the use of liquid nitrogen (LN₂), because commonly used cryopreservation media containing cell membrane permeating cryoprotectants are thermally unstable when frozen at non-cryogenic temperatures, e.g. −80° C., thereby leading to ice recrystallization and causing progressive loss of cell viability over time under the storage conditions provided by most laboratory deep freezers. The dependency on LN₂ for cell storage significantly increases operational expense and raises numerous issues related to impaired working efficiency and safety.

Two general approaches are widely used in cryopreservation: equilibrium (slow freezing) and non-equilibrium (vitrification) cooling procedures. The vitrification method, as well as its “slow vitrification” variant, not only introduces cell osmotic damage and toxicity due to the use of high concentrations (typically 40-50% v/v) of permeating cryoprotectant but requires a supply of LN₂ or other cryogenic liquids to achieve and maintain vitrification of both intracellular and extracellular solutions at cryogenic temperatures, e.g. the saturation temperature of LN₂ at one atmosphere pressure (−196° C.) or LN₂ vapor (typically −120° C.). For the slow freezing approach, cells are first loaded with a relatively low concentration (typically 10% v/v) of cryoprotectant and then slowly cooled to an intermediate non-cryogenic temperature, e.g. −80° C. in a deep freezer. During cooling, ice precipitation gradually increases solute concentrations, such that, after reaching the intermediate temperature, the residual solution containing the cells is highly concentrated and in a viscous liquid state. The extracellular ice in such a partially frozen system is unstable, and the small ice crystals formed during cooling spontaneously begin to merge and form larger crystals to minimize their surface energy and become progressively distributed throughout the sample. Such events, so-called recrystallization, either cause severe mechanical damage to cells that contact the emerging large crystals or introduce lethal intracellular ice formation. Even though this cell damaging process is quite slow (typically occurring over weeks rather than hours), it is progressive even at temperatures as low as −80° C. Demonstrated by numerous publications, in either scientific research articles or cryopreservation medium product manuals, current storage in −80° C. deep freezers is only suitable for temporary or short-term purposes of use. Accordingly, to achieve long-term storage of cells after they have been slowly frozen at −80° C., it has been necessary to have a second step in which the samples are cooled to cryogenic temperatures.

Antifreeze proteins and certain small molecules are able to quench ice recrystallization by inducing thermal hysteresis, but this process only occurs over a temperature range just below the melting point of ice and is ineffective at lower temperatures.

Various polymers and associated methods have been developed to improve post-thaw cell viability and functionality by increasing solution viscosity or improving cell membrane stability after storage in liquid nitrogen. However, currently, there has been no significant improvement on the low survival rate of many cell types that are highly valuable for both research and biomedical applications after long-term storage at −80° C. by using these polymers and methods. The application of these widely used polymers for cryopreservation, e.g. polyvinylpyrrolidone (PVP), Hydroxyethyl starch (HES), etc are inadequate in prevention of recrystallization of cryoprotectant solutions at non-cryogenic temperatures.

### BRIEF SUMMARY OF THE INVENTION

The present invention is directed to media for preserving cells at non-cryogenic freezing temperatures comprising a macromolecule that forms compact three-dimensional structures that are spherical in shape when dissolved in an aqueous liquid. The invention is also directed to medium-cellular suspensions comprising a medium of the present invention with the cells suspended in the medium. The invention is further directed to methods of using the medium of the present invention to preserve cells. At non-cryogenic freezing temperatures the compact and spherical structures are concentrated in an unfrozen portion of the medium with the cells and this crowding effect prevents ice recrystallization during storage at non-cryogenic temperatures.

In certain aspects of the invention, the medium comprises a hydrophilic and nontoxic macromolecule, an aqueous liquid, and a cryoprotectant. The macromolecule may be at a concentration in the medium equal to or greater than about 20% (w/v), about 25% (w/v) or greater, about 35% (w/v) or greater or about 50% (w/v) or greater.

In certain aspects of the invention, the cryoprotectant is at a concentration equal to or greater than about 20% of the concentration of the macromolecule in the medium, equal to or greater than about 50% of the concentration of the macromolecule in the medium, equal to or greater than about 75% of the concentration of the macromolecule in the medium or equal to or greater than about 100% of the concentration of the macromolecule in the medium.

In certain aspects of the invention the macromolecule is a polymer. The polymer may comprise molecules that form the compact three-dimensional structures that are approximately spherical in shape when dissolved in the aqueous liquid. In such embodiments, the polymer may be selected from the group consisting of spherical hydrophilic polysaccharides, polymerized cyclodextrin or saccharides, globular proteins or spheroproteins, spherical glycoproteins formed by attaching oligosaccharide chains to those globular proteins, other derivatives of those globular proteins or combinations thereof. The polymer may be a hydrophilic polysaccharide and may be formed by the copolymerization of sucrose and epichlorohydrin.

In certain aspects of the invention, the cryoprotectant is selected from the group consisting of dimethyl sulphoxide (DMSO), glycerol, ethylene glycol, propanediol, and combinations thereof. In certain aspects of the invention, the aqueous liquid is selected from the group consisting of a cell culture medium, a nutritious medium, a saline and combinations thereof. The aqueous liquid may be selected from the group consisting of serums, FBS (fetal bovine serum), DMEM (Dulbecco's Modified Eagle Medium), HEPES (4-(2-hyroxyethyl)-1-pierazineethanesulfonic acid), FHM (flushing-holding medium), PBS (phosphate-buffered saline), DPBS (Dulbecco's phosphate-buffered saline), RPMI (Roswell Park Memorial Institute medium), BF5 medium, EX-CELL medium, Lysogeny broth (LB) medium, CaCl₂ aqueous solution, NaCl aqueous solutions, KCl aqueous solutions and combinations thereof.

In certain aspects of the invention, the suspended cells are eukaryotic cells. The eukaryotic cells may be mammalian cells. The mammalian cells may be selected from the group consisting of murine cells, porcine cells, human cells, and combinations thereof. The mammalian cells may be selected from the group consisting of stem cells, somatic cells, reproduction cells and combinations thereof. In other aspects of the invention, the suspended cells are prokaryotic cells.

In certain aspects of the invention, the compact approximately spherical structures are about 100 nm (nanometer) or less in their widest dimension, comprise structures ranging from about 1 to 50 nm in their widest dimension or comprise structures ranging from about 5 nm to 10 nm in their widest dimension.

In certain aspects of the invention, the medium is substantially free of serum, animal proteins or human proteins.

Certain aspects of the invention are directed to a method for preserving cells at non-cryogenic freezing temperatures that includes providing a cryopreservation medium comprising a hydrophilic and nontoxic macromolecule, a cryoprotectant, and an aqueous liquid. In certain embodiments, the macromolecule is at a concentration in the medium greater than 10% (w/v), and the macromolecule forms a highly compact approximately spherical structure when dissolved in the aqueous liquid. The cells are added to the medium to form a medium-cellular suspension. The medium-cellular suspension is cooled to a non-cryogenic freezing temperature, wherein the non-cryogenic freezing temperature is about −85° C. or higher. The medium-cellular suspension may be maintained at or near the non-cryogenic freezing temperature, or a different non-cryogenic freezing temperature, for a time period longer than three weeks while maintaining post-thaw cell survival rates of the cells equal to or about the same as would be obtained for storage of the cells in liquid nitrogen for the same period of time. In certain aspects of the method, the macromolecule is a polymer.

In certain aspects of the method, the concentration of the polymer or other macromolecule in the cryopreservation medium ranges from 10% to the polymer's solubility in the aqueous liquid, or ranges from 20% to 50%.

In certain aspects of the method, the cells added to the medium are in a first suspension of cells, wherein the volumetric ratio of the cryopreservation medium to the first suspension of the cells is from 10:1 and 1:5, or is from 3:2 to 1:5.

In certain aspects of the invention, the medium-cellular suspension is stored for a time period of about three weeks and extending up to at least one year while maintaining post-thaw cell survival rates of the cells equal to or about the same as would be obtained for storage of the cells in liquid nitrogen for such period of time. In certain embodiments the time period is one year or more, 5 years or more, or 10 years or more.

In certain embodiments, the post-thaw cell survival rate is equal to or greater than about 70% of that obtained for storage of the cells in liquid nitrogen for the same period of time.

In certain embodiments, the non-cryogenic temperature ranges from −100° C. to −20° C., ranges from −85° C. to −65° C., or ranges from −80° C. to −75° C.

In certain embodiments, the medium-cellular suspension is cooled at a rate of about 0.01° C./min to 1000° C./min, a rate of about 0.1 to 10° C./min, or a rate of about 0.5 to 1° C./min.

In certain embodiments, after the cooling step, the medium-cellular suspension is partially frozen and the macromolecule is at a concentration of at least 25% (w/v) in an unfrozen portion of the medium-cellular suspension, or is at a concentration of at least 40% (w/v).

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### DETAILED DESCRIPTION OF PREFERRED EMBODIMENT

The present invention is directed to a medium for preserving cells at non-cryogenic freezing temperatures. The medium comprises a hydrophilic and nontoxic polymer or other macromolecule, an aqueous liquid, and a cryoprotectant. The molecules of the polymer or other macromolecule form compact three-dimensional structures that are spherical in shape when dissolved in the aqueous liquid.

As described in more detail in the Examples, it was surprisingly found that when cells are suspended in the medium of the present invention, the resulting mixture, also referred to herein as the medium-cellular suspension, can be stored at non-cryogenic freezing temperatures for unexpectedly long periods of time with results similar to those obtained with storage at cryogenic temperatures with liquid nitrogen. It is believed this is due to a macromolecular crowding effect discovered to result from the highly compact and mechanically strong three-dimensional structure formed by the macromolecules in the medium of the present invention. At a non-cryogenic freezing temperature the three-dimensional structures also occupy a large portion of, or are highly concentrated in, the unfrozen portion of the medium of the medium-cellular suspension. The unfrozen portion of the medium is in phase equilibrium with ice crystals formed during freezing, along with the cells, and this crowding effect prevents ice recrystallization during storage at the non-cryogenic freezing temperatures. In certain embodiments, the concentration of the polymer or other macromolecule in the unfrozen portion of the medium of the medium-cellular suspension is at least about 25% (w/v), at least about 35% (w/v), at least about 40% (w/v) or any value or range therein.

This newly discovered macro-molecular crowding effect is supported by the simulation described in more detail in Example 1. As shown in FIG. 1, the simulation demonstrated that when a medium of the present invention is cooled to −80° C., a unique network of many individual highly compact and mechanically strong macromolecule spheres is formed as a mechanical barrier that quenches the growth of ice nuclei. As shown in FIG. 2, the presence of the polymer results in a lower RMS distance of atomic positions, indicating a higher thermal stability of water molecules around an ice nucleus artificially placed in the simulation box. Until discovery of this macromolecular crowding effect, it would not have been expected that a cryopreservation medium could maintain cell viability at non-cryogenic temperatures similar to that of cryogenic freezing over long periods of time.

Example 2 provides scanning electron microscopy (SEM) evidence of the lowered ice recrystallization resulting from the macromolecular crowding effect. As shown in FIG. 3, a medium of the present invention preserves the ice crystal morphology as granulates, and single ice crystals are readily identified (FIG. 3(B)). This is in contrast to the merged large blocks or sheets of ice crystals in a 10% DMSO medium (FIG. 3(A)). This is supported by a simple optical observation, as shown in FIG. 4, comparing (A) the control and (B) a medium of the present invention.

The macromolecule of the present invention may be any hydrophilic and nontoxic macromolecule that forms a compact three-dimensional structure that is spherical in shape when dissolved in the aqueous liquid. The compact structures are preferably about 100 nm (nanometer) or less in their widest dimension. In certain embodiments, the compact structures include structures ranging from about 1 to 50 nm in their widest dimension, from about 5 to 10 nm in their widest dimension, or any value or range therebetween. It should be understood that not all of the macromolecules contained in the medium must be within the desired ranges. The term “spherical” does not require that the macromolecules form structures that are a perfect sphere. Rather the macromolecules form structures that are generally spherical in shape.

In certain embodiments, the macromolecule is a polymer. The polymer may be a hydrophilic polysaccharide or similar structured macromolecule having molecules that form compact three-dimensional structures that are spherical in shape when dissolved in an aqueous liquid. Suitable macromolecules include spherical hydrophilic polysaccharides, polymerization of cyclodextrin or any saccharides to form large spherical molecules, globular proteins or spheroproteins (e.g. albumins, such as bovine serum albumin (BSA)), spherical glycoproteins formed by attaching oligosaccharide chains to those globular proteins or other derivatives of those globular proteins. Hydrophilic nanoparticles can also be suitable macromolecules. One suitable polymer is a polymer formed by the copolymerization of sucrose and epichlorohydrin, without any ionized groups, such as that sold under the brand name FICOLL by GE Healthcare Bio-Sciences AB.

In certain embodiments the macromolecule has a molecular weight from about 300,000 to about 500,000, preferably 400,000, such as that sold under the brand name Ficoll 400. In other embodiments, the macromolecule has a molecular weight from about 60,000 to about 80,000, preferably about 70,000, such as that sold under the brand name Ficoll 70.

In certain embodiments of the medium, before addition of cells, the polymer or other macromolecule is present at a concentration greater than about 10% (w/v) or greater, about 20% (w/v) or greater, about 25% (w/v) or greater, about 35% (w/v) or greater, or about 50% (w/v) or greater, or any range or value therein. In certain embodiments, the polymer or other macromolecule is at a concentration up to the solubility of the polymer (or other macromolecule) in the aqueous liquid or water.

The cryoprotectant can be any cryoprotectant known in the art. Preferably the cryoprotectant is a cell permeating small organic molecule. Cryoprotectants suitable for use in the present invention include dimethyl sulphoxide (DMSO), glycerol, ethylene glycol, propanediol, and combinations thereof.

The medium of the present invention allows use of lower amounts of the cryoprotectant to be used than in standard cryopreservation media. The cryoprotectant can be present in the medium at a concentration equal to or greater than about 20% of the concentration of the polymer (or other macromolecule) in the medium, equal to or greater than about 50% of the concentration of said polymer in the medium, equal to or greater than about 75% of the concentration of said polymer in the medium, or equal to or greater than about 100% of the concentration of said polymer in the medium, or any value or range therein. The volumetric ratio of the polymer (or other macromolecule) and aqueous liquid to the cryoprotectant is from 10:1 to 1:1, from 5:1 to 1:1, or any range or values therebetween.

The aqueous liquid can be any aqueous liquid suitable for use in suspending cells, and can be a liquid selected from the group consisting of a cell culture medium, a nutritious medium, a saline and combinations thereof. Aqueous liquids suitable for use with the present invention include serums, FBS (fetal bovine serum), DMEM (Dulbecco's Modified Eagle Medium), HEPES (4-(2-hyroxyethyl)-1-pierazineethanesulfonic acid), FHM (flushing-holding medium), PBS (phosphate-buffered saline), DPBS (Dulbecco's phosphate-buffered saline), RPMI (Roswell Park Memorial Institute medium), BF5 medium, EX-CELL medium, Lysogeny broth (LB) medium, CaCl₂ aqueous solution, NaCl aqueous solutions, KCl aqueous solutions and combinations thereof. The medium may be substantially free of serum, animal proteins or human proteins.

The medium of the present invention is suitable for use with any types of cells. For example, the suspended cells can be eukaryotic cells. The eukaryotic cells may be mammalian cells, such as mammalian cells selected from the group consisting of murine cells, porcine cells, human cells, and combinations thereof. The mammalian cells can be any type of cell, including cells selected from the group consisting of stem cells, somatic cells, reproduction cells and combinations thereof. Reproduction cells may include, for example, embryos and oocytes. Other eukaryotic cells that may be used included insect cells. In other embodiments the calls may be prokaryotic cells. The prokaryotic cells may be bacteria, such as E. coli, Streptococcus and Staphylococcus.

The cells may be separated into single cells or may be in clumps. Cells may be added as isolated cells or in a suspension. The term “cells” may also encompass other cellular materials comprising multiple cells, including tissues.

For suspensions of mammalian cells, their cell concentration may be in the range of 10⁵ to 10⁶ cells per 0.5-1 ml cell suspension sample. For oocytes and embryos, the cell density (number) is low, typically around 10² to 10⁵ cells in one sample (sample volume is around 0.25-0.5 ml), because of the difficulty in obtaining millions of embryos or oocytes. In certain embodiments, it may be possible to preserve only several hundred embryos or oocytes in one sample (e.g. a 0.5 ml straw containing embryos or oocytes), and in some embodiments, the number of embryos or oocytes in the sample is around 20. Prokaryotes, e.g. E. coli, can be available in high density. Prior to freezing, the cell concentration can reach 10⁹⁻¹⁰ cells per ml.

The present invention is also directed to methods for preserving cells at a non-cryogenic freezing temperature in a medium of a present invention. When used herein, the term non-cryogenic freezing temperature can be any temperature above the saturation temperature of LN₂ at one atmosphere pressure (−196° C.) or LN₂ vapor (typically −120° C.). Non-cryogenic freezing generally occurs in freezer set at −80° C., with temperatures that can go as low as −85° C., but can also rise above −80° C. due to temperature variations that can result from opening the freezer door or placing unfrozen materials into the freezer. The medium of the present invention also allows the cells to be maintained frozen by dry ice while maintaining acceptable cell survival rates. Suitable non-cryogenic freezing temperatures can include temperatures from about −100° C. to −20° C., about −85° C. to −65° C., or about −80° C. to −75° C., and any values and ranges therebetween.

The process includes providing a cryopreservation medium comprising a hydrophilic and nontoxic macromolecule, a cryoprotectant, and an aqueous liquid, wherein the macromolecule forms a highly compact spherical structure when dissolved in said aqueous liquid, adding said medium to cell suspensions, or adding cells or cell suspensions to said medium, or in any order of adding part of said medium or cell suspension, to form a medium-cellular suspension, and cooling the medium-cellular suspension to a non-cryogenic freezing temperature.

The total concentration of cells in the medium-cellular suspension prior to freezing can vary widely depending on the intended use, as will be readily understood to those in skilled in the art. In certain embodiments, the concentration of cells in the cryopreservation medium prior to freezing is single or sparsely distributed cells in the whole system, 10²⁻⁴ cells/ml, 10⁵⁻⁶ cells/ml, 10⁷ or more cells/ml, or even a whole tissue or any value or range therebetween. In certain embodiments, the cells are added as cellular suspension, and the volumetric ratio of the cryopreservation medium to the suspension of cells can range from about 10:1 to about 1:5, about 2:1 to about 1:2, about 3:2 to 1:1, or any value and range therebetween.

The cooling step will generally involve slow cooling. In such embodiments, the medium-cellular suspension can be cooled at a rate of about 0.01° C./min to about 1000° C./min, about 0.1 to about 10° C./min, about 0.5 to 1° C./min, or any value or range therebetween.

As discussed in more detail above and in the examples, the polymer or other macromolecule is concentrated in an unfrozen portion of the medium-cellular suspension after the cooling step. In certain embodiments, the concentration of the polymer or other macromolecule in the unfrozen portion of the medium is at least about 25% (w/v), at least about 35% (w/v), at least about 40% (w/v) or any value or range therein.

The medium-cellular suspension can be maintained at a non-cryogenic freezing temperature for long periods of time. It should be understood that although the medium-cellular suspension can be maintained at or near the original non-cryogenic freezing temperature for the entire time it is frozen, the temperature can vary between different non-cryogenic freezing temperatures during the freeing period. During storage, the medium-cellular suspension can also be cooled to cryogenic freezing temperatures for periods of time and warmed up back to non-cryogenic temperature range for the rest of the period of time (e.g. in the case that cells are stored in liquid nitrogen by one user and then stored in deep freezers by another user; or cells are stored in liquid nitrogen, but warmed and shipped in dry ice box (above −78° C.)).

As discussed above, the medium-cellular suspension can be maintained at a non-cryogenic freezing temperature for surprisingly long periods of time, while maintaining post-thaw cell survival rates of the cells about the same as would be obtained for storage of the cells in liquid nitrogen for the same period of time. Consistent with the present invention, the medium-cellular suspension can be maintained at non-cryogenic freezing temperatures for over three weeks, about three weeks and extending up to at least one year, about one year or more, about 5 years or more, or about 10 years or more, and any time period or range of time periods therein.

As demonstrated in the Examples, at the end of the freezing period, the cells stored in the medium-cellular suspension have a post-thaw cell survival rate about the same as would be obtained for storage of said cells in liquid nitrogen for the same period of time. The cell survival rate can be at least about 80%, at least about 90%, about 100% or higher, and any value or range therebetween, of the cell survival rate for cells stored in liquid nitrogen for the same period of time. Without using the medium of the present invention, for example using solely 10% DMSO as the cryoprotectant, 5% to 30% (of the number of those survived from storage in liquid nitrogen) cells (depending on cell types, see FIGS. 6. A, B and D) survive after about 2-3 months of storage at −80° C., about 0% survive after one year storage (as shown in FIG. 6.A, 58 weeks), Thus the cell survival rate for cells stored using the medium of the present invention is significantly higher than that of cells stored at −80° C. without using the medium of the present invention.

The polymer (or other macromolecule) and cryoprotectant can be combined with each other and the liquid in any order that allows the polymer to be dissolved in the desired concentration. In certain embodiments, the polymer is first dissolved in the aqueous liquid to form a first mixture, and the cryoprotectant is then added, or that order can be reversed. In other embodiments, the cryoprotectant and polymer are added simultaneously or small amounts of each can be added until the desired ratios are reached. The volumetric ratio of the Ficoll/aqueous liquid to cryoprotectant can range from about 10:1 to 1:5, about 5:1 to 1:1, about 2:1 to 1:1, or any values or ranges therebetween.

The present invention demonstrates that addition of a hydrophilic and nontoxic macromolecule of the present invention to typical cryopreservation solutions significantly improves system thermal stability at non-cryogenic freezing temperatures. It is believed this occurs through macromolecular crowding effects achieved by the macromolecule after slow freezing procedures. Accordingly, using the cryopreservation medium of the present invention provides reliable cryopreservation of various kinds of cells at −80° C. for at least one year, with the post-thaw viability, plating efficiency, and full retention of cell phenotype comparable to that achieved with LN₂ storage. These results achieved with the medium of the present invention illustrate the practicability of a non-cryogenic cell storage method that completely eliminates the need of LN₂.

Certain aspects of the present invention are illustrated by the following non-limiting examples.

### Example 1. Molecular Dynamic Study Demonstrating the Macromolecular Crowding Effects of Compact 3-D Structured Hydrophilic Polysaccharide Molecules in Preventing Ice Recrystallization

Three simulation boxes for molecular systems including Ficoll 70-DMSO-water, sucrose-DMSO-water and DMSO-water were prepared for the molecular dynamics simulations. For all these systems, the systematic temperature was fixed as −80° C. The concentration of each component is determined by premeasured phase diagram of these ternary systems. For the Ficoll 70-DMSO-water system with the Ficoll and DMSO mass ratio as 1:1, the phase diagram determines that at −80° C., the concentrations of Ficoll, DMSO and liquid water are to be 35%, 35% and 30% (w/w) to reach phase equilibrium with solid water (ice phase). In other words, for the medium of the present invention, after it mixes with DMSO and a cell suspension, the new mixture is slowly cooled to −80° C., and the Ficoll concentration is then significantly increased. FIG. 1(A) depicts the molecular dynamic demonstration of the macromolecular crowding behavior of a Ficoll-DMSO-water system at −80° C., with an ice nucleus placed at the center to test the systemic stability. The left photo is the whole simulation box. The right photo is the cross sectional view of the system to show the ice nucleus. FIG. 1(B) depicts the molecular dynamic demonstration of the evenly distributed sucrose-DMSO-water system at −80° C., with an ice nucleus placed at the center to test the systemic stability. The left photo is the whole simulation box. The rights photo is the cross sectional view of the system to show the ice nucleus. FIG. 1(C) depicts the molecular dynamic demonstration of the evenly distributed DMSO-water system at −80° C., with an ice nucleus placed at the center to test the systemic stability. The left photo is the whole simulation box. The rights photo is the cross sectional view of the system to show the ice nucleus.

Importantly, as demonstrated in FIG. 1(A), a unique network of many individual Ficoll spheres (˜5 nm in diameter) is formed.

According to phase diagrams, for the sucrose-DMSO-water system with sucrose and DMSO weight ratio as 1:1, the sucrose, DMSO, and water concentrations are 36%, 36% and 28% (w/w), respectively, when they reach phase equilibrium with the ice phase; for the DMSO-water system with DMSO and water weight ratio as 1:9 prior freezing (as in widely used cryopreservation methods), their phase equilibrium concentrations are 58% and 42% respectively. The dimension of the simulation boxes of these three cases, Ficoll-DMSO-water (FIG. 1(A)), sucrose-DMSO-water (FIG. 1(C)), and DMSO-water (FIG. 1(C)) are 156.929×159.481×159.300 Å, 163.532×169.882×159.953 Å and 140.624×145.061×135.631 Å, while the total atom number are 235,440, 242,118 and 175,744, respectively.

To demonstrate the thermal stability of above systems, a typical cubic ice nucleus, presented as a group of 512 water molecules forming a 10 nm cube, is artificially placed at the center of each simulation box, as shown in FIG. 1, simulating the case that ice nucleation is initiated but not further developed in the above unfrozen solutions during either freezing or storage. The stability of this ice nucleus in these three different systems was analyzed through molecular dynamics. The simulations were performed with the commonly used Large-scale Atomic/Molecular Massively Parallel Simulator (LAMMPS) which is distributed by the Sandia National Laboratories. The VMD (visual molecular dynamics) was used to visualize the data as well as the data processing. Before the simulations were conducted, all these molecular systems were performed with an energy minimization procedure in order to find a better initial starting configuration. Once the potential minimized structures were found, the entire systems were equilibrated at a state that own fixed density and temperature desired. The entire simulations were performed with 15,4000 for equilibrium and another 504,000 steps for data sampling with the same time step of 1 fs.

The activity of the liquid water molecules surrounding the ice nucleus was measured through the root-mean-square (RMS) distance of atomic positions, along with the final equilibrated structures of these three systems, and the results are shown in FIG. 2, in which Ficoll-DMSO-Water is the bottom curve, sucrose-DMSO-water is in the middle curve, and DMSO-water is in the top curve. It is demonstrated that the system of DMSO-Water yields the largest value of RMS distance, demonstrating the lowest thermal instability. The Sucrose-DMSO-Water system shows a slightly lowered value of RMS distance. The system with presence of the Ficoll has significantly lowered value of RMS, and in other words, results in highest thermal stability of water molecules around the nucleus. Therefore, it can be concluded that the chance of the direct interaction between liquid water molecules and the ice nucleus can be significantly attenuated by the presence of Ficoll spheres, and this mechanism results in the prevention of ice recrystallization at −80° C. Due to the amorphous structures of the molecules including PVP, PEG, HES, PVA, etc, it is technically difficult to include them into the molecular dynamic model, but it can be predicted that their rather evenly distributed linear molecules or resulting loose 3-D structure would result in similar values of RMS distance compared to those of the sucrose-DMSO-water system, and result in much lower thermal stability compared to the system with Ficoll highly compacted spherical molecules.

### Example 2. Prevention of Recrystallization of Cryopreservation Solutions at Non-Cryogenic Temperatures

Scanning electron microscopy (SEM) was performed to demonstrate the capability of a medium of the present invention in significantly improving the thermal stability of ice crystals in samples.

In one embodiment of the medium of the present invention, a solution of 20% (w/v) Ficoll 70 and 20% (v/v) DMSO in DMEM medium was gradually mixed with a substitute of cell suspension, e.g. DMEM medium without any cells, with a volume ratio of 3:2, and transferred to 1.5 ml cryovials. These vials were then loaded into a widely used freezing container (Nalgene Mr. Frosty), which was mounted into a −80° C. freezer to provide an approx. 1° C./min cooling rate until the next day. These vials were then transferred into a precooled and sealed vial container in the freezer and stored for 5 weeks. For comparison, the control group as the solution of 10% (v/v) DMSO in DMEM medium was also cooled and stored in the same approach and for the same period of time. At the end of 5 weeks, the cryovials were directly plunged into LN₂ to fix the ice crystal morphology and were fractured inside LN₂. The fixed and fractured samples covered by LN₂ in a copper stage and were then transferred into the vacuum chamber of a SEM system. After the LN₂ evaporated and chamber pressure was lower than 10⁻² Pa, the surface of the samples were scanned by the SEM to observe the ice morphology for the fracture surface. FIG. 3 shows the SEM observation of fractured samples after 5 week storage, with the same amplification and bar length as 500 μm. FIG. 3A shows the SEM observation of the normal frozen cryopreservation solution, and FIG. 3B shows the SEM observation of a medium of the present invention incorporating the same cryoprotectant. As shown in FIG. 3.A, for the control solution containing only 10% DMSO, ice crystals have merged into large blocks or sheets after 5 week storage, and it is difficult to identify any single ice crystal. In contrast, as shown in FIG. 3.B, the medium of the present invention preserves the ice crystal morphology as granulates and single ice crystals are readily identified. As shown in FIG. 4, simple optical observation of samples stored five weeks supported the same conclusion: the control solution (10% DMSO and 90% DMEM) (A) is opaque (more white in color in the vial show in the picture) due to the large size of the recrystallized ice, while the mixture of the medium of the present invention with DMEM (B), as described above, resulted in a more transparent (i.e. less white) frozen solution due to less quantity and smaller size of ice crystals.

### Example 3. Use of Differential Scanning Calorimetry to Examine Thermal Stability

Crowding of Ficoll, a compact, spherical, hydrophilic polysaccharide, in aqueous solutions can restrict microscale diffusion and limit structural reconfiguration of macromolecules. To explore its potential in regulating ice recrystallization, differential scanning calorimetry (DSC) was used to examine the thermal stability of aqueous solutions of DMSO and Ficoll (Ficoll 70 and Ficoll 400) at non-cryogenic temperatures. For comparison, similar ternary systems containing DMSO and polyvinylpyrrolidone (PVP) or sucrose, representing commonly used polymers and small molecules used for cryopreservation, were also tested. To model the residual solutions at the end of a slow freezing process, highly concentrated solutions comprised of a 1:1 weight ratio of one of the non-permeating solutes and the permeating cryoprotectant DMSO were prepared with the same total solute concentration (50% w/w). A standard DSC procedure to detect vitrification and devitrification was followed using a Pyris Diamond DSC (Perkin-Elmer Corp). A volume of 8 μl of each model solution was sealed in a standard 10 μl aluminum crucible (Perkin-Elmer Corp) designed for liquid samples and then loaded in the sample holder of the DSC machine. All samples were cooled to −160° C. from 1° C. at 100° C./min to achieve complete vitrification, which was confirmed by continuous heat capacity change near −130° C. for all the samples during the cooling and following warming procedures, and none of the samples experienced any crystallization during the cooling process. After being held at −160° C. for 1 min, the samples were heated to 20° C. at a warming rate of 10° C./min. Devitrification was detected in all the samples and the onset temperatures for the corresponding exothermic curves were determined as the values of devitrification temperatures using the Pyris™ thermal analytic software provided by Perkin-Elmer Corp. Table 1 shows the devitrification temperatures (Td) of the highly concentrated solutions modeling the unfrozen residual portion of the aqueous solutions containing one polymer (or sucrose) and DMSO at the end of a slow freezing process. The total solute weight percentage for each solution is fixed as 50% w/w.

TABLE 1

| Solute concentrations (w/w) | T_{d} | |
|---|---|---|
| 25% Ficoll 70, 25% DMSO | −67.0° | C. |
| 25% Ficoll 400, 25% DMSO | −75.7° | C. |
| 25% PVP 40, 25% DMSO | −91.5° | C. |
| 25% PVP 360, 25% DMSO | −101.8° | C. |
| 25% sucrose, 25% DMSO | −110.1° | C. |
| 50% DMSO | −118.2° | C. |
| 16.7% Ficoll 70, 33.3% DMSO | −90.6° | C. |

Since recrystallization involves a spontaneous process that generates no detectable latent heat release, the thermal stabilities of these model solutions were assessed by their devitrification temperatures (Td). This approach is possible because Td is always lower than, but close to, the temperature at which recrystallization begins. Accordingly, if the Td measured for any of these model solutions during slow warming is higher than −80° C., as observed for solutions containing Ficoll 70 (Td, −67° C.) or Ficoll 400 (Td, −75.7° C.), it is considered to be thermally stable and no recrystallization will occur at −80° C. (Table 1). Of the two Ficoll polymers, Ficoll 70 appeared to be superior at providing a potentially useful cryopreservation medium, since at a 1:1 weight ratio with DMSO it demonstrated the higher Td value. These results suggested that initiating slow freezing with lower concentrations (e.g. 10% for each) of Ficoll 70 and DMSO, to achieve sufficient macromolecular crowding by cooling to −80° C. when the cryopreservation medium is much more concentrated, potentially prevents recrystallization in extracellular solutions and hence realize long-term storage of cells at −80° C.

### Example 4. Examination of Viability and Pluripotent Features of O2K Porcine iPSC Cells after Long-Term Storage at −80° C.

The ability of the medium of the present invention to preserve the viability and pluripotent features of the O2K line of porcine induced pluripotent stem cells (iPSC) during long-term storage in a commercial deep freezer was examined. The O2K line of porcine iPSC is a naïve-type of pluripotent stem cell, dependent upon leukemia inhibitory factor (LIF) and STAT3 signaling for self-renewal, which can be dispersed into single cells without significant loss of viability. For routine maintenance, O2K piPSC were cultured either on a laminin (Gibco) coated substratum or irradiated mouse embryonic fibroblasts feeder on six-well culture plates (Nunc) in N2B27 (Gibco) medium, supplemented with three inhibitors (CHIR99021 (Stemgent), PD032591 (Stemgent), and PD173074), 2 μg/ml doxycycline (Stemgent), and 1000 unit/ml human LIF (Millipore). O2K piPSC were passaged every three days after dispersing with Accutase (Millipore) for 7 min at 37° C. Cell colonies were dispersed to single cells with a cell detachment solution sold under the tradename Accutase® by Innovative Cell Technologies, Inc. Dissociated cells were collected by centrifugation (200×g for 5 min) and resuspended in chilled culture medium. Different embodiments of the medium of the present invention were prepared as: 10% (w/v) Ficoll 70 and 20% (v/v) DMSO, 20% (w/v) Ficoll 70 and 20% (v/v) DMSO, 30% (w/v) Ficoll and 20% (v/v) DMSO. These media were based on either FBS (a serum) or Dulbecco's Modified Eagle Medium (DMEM/F12, serum-free), and in another word, excluding Ficoll 70 and DMSO, the liquid portion of the media is either FBS or DMEM. Each of these media was added drop-wise to a suspension of cells in their culture media (total volume ratio between the cell suspension and all added medium drops is approximately 3:2). After mixture, the final concentration in the medium-cellular suspension of DMSO was approximately 10% v/v and Ficoll 70 as 5%, 10% or 15% w/v prior to freezing, respectively. Such a mixing procedure is to modify the cell damage caused by osmotic damage generated by directly mixing the whole cryopreservation media with cell suspensions. The cryovials were then placed into a freezing box (Mr. Frosty, Nalgene), as widely used for current cryopreservation of many cell types. The latter was placed overnight into a −80° C. freezer to provide an approximately 1° C./min cooling rate. On the following day, the vials were stored in the −80° C. freezer for two weeks. The control groups were cells treated with a similar procedure to achieve the same final concentration of DMSO (10%), except that the cryopreservation medium was based on FBS alone and contained no Ficoll, as generally used for stem cell LN₂ storage. These control samples were cooled by the same slow freezing procedure and then stored at −80° C. (as a negative control) or in a LN₂ dewar (as a positive control).

For thawing, all cryovials were rapidly warmed in a 37° C. water bath for approximately 1 min until the ice mass disappeared. The medium-cellular suspension was then transferred to a 15 ml centrifuge tube and slowly mixed with 5 ml of warmed culture medium. After centrifugation (200×g for 5 min), the supernatant solution was removed, and cell pellets resuspended in 1 ml fresh culture medium.

Thawed and cultured cells were plated in a 6-well plate and cultured overnight. After the first medium change, images of adherent colonies were acquired over five different areas within each well. Plating efficiency was estimated as colonies/number of initially plated cells×100%. Colonies were then fully dispersed by Accutase, and total cell numbers were assessed by using a TC10 automated cell counter (Bio-Rad).

The results after two weeks of storage are shown in FIG. 5 for (A) FBS-based media and (B) serum-free DMEM/F12 based media. Within each figure, bar values are means±SEM (n=3), with different letters (a, b, c) indicating significantly different (P<0.05) values.

Only the cells cryopreserved at −80° C. within the medium-cellular suspension comprising the mixture of cell suspension and the 20% Ficoll and 20% DMSO medium (final post-mixture and prior-freezing Ficoll concentration is 10% as shown in FIG. 5 and explained above) provided a plating efficiency comparable to that of the LN₂ storage control. The lower and the higher concentrations of Ficoll afforded significantly worse survival. The presence of FBS did not affect outcomes under any of the freezing conditions tested. The 20% Ficoll 70 and 20% DMSO medium was then used to mix with cell suspensions for the following longer term storage and for other cell types.

The results for O2K porcine iPSC thawed after 2, 5, 10 and 58 weeks are shown in FIG. 6A. FIG. 6 shows results for cells cryopreserved under three conditions: 10% v/v DMSO and stored in LN₂ shown as the left bars, 10% v/v DMSO and stored in a −80° C. freezer shown in the center bar, and medium-cellular suspension comprising the mixture of the cell suspension with the 20% Ficoll 70 and 20% DMSO medium stored in a −80° C. freezer in the right bar. Bar values are means±SEM (n=3), with different letters (a, b) indicating significantly different (P<0.05) values within the results on the same checking point.

Even by the end of week 2, the ability of the cells cryopreserved without using the medium of present invention at −80° C. to attach, proliferate and provide colonies (red bars) had fallen significantly relative to the other two treatments. These declines were progressive over storage time, such that, at 10 weeks, recovery was very low, and no colonies at all formed after 58 weeks of storage, results consistent with the concept that the recrystallization process causes progressive rather than immediate cell damage. By contrast, the porcine iPSC stored at −80° C. within the mixture of cell suspension and said embodiment of the medium of the present invention showed no apparent decrease in either plating efficiency (FIG. 6A, left panel) or proliferative capacity (FIG. 6A, right panel) relative to LN₂ storage over time.

For the tests of pluripotency of the thawed cells, after thawing, cells were allowed to establish colonies, passaged and grown on coverslips. Specimens were fixed in 4% v/v paraformaldehyde in PBS for 15 min at room temperature, washed, and exposed to either 5% v/v goat serum or 5% v/v donkey serum, 1% w/v bovine serum albumin, and 0.1% v/v Triton X-100 (Fisher) in PBS for 30 min. The fixed specimens were then incubated with primary antibodies at 4° C. overnight. After washing, they were exposed to secondary antibodies. Colonies exposed only to secondary antibody served as controls. VECTASHIELD mounting medium with DAPI (Vector Laboratories) was used to mount the coverslips. Primary antibodies were: POU5F1 (1:100, Santa Cruz Biotechnology), SOX2 (1:1000; Millipore), NANOG (1:200; Abcam), SSEA1 (1:50; Developmental Studies Hybridoma Bank [DSHB]). As shown in FIG. 9A, the cells from −80° C. storage using the method of present invention also retained a pluripotent phenotype.

### Example 5. Examination of Viability and Pluripotent Features of ID6 Porcine iPSC Cells after Long-Term Storage at −80° C.

The morphology of ID6 porcine iPSC during culture is shown in FIG. 7A. The cells form flat, adhesive colonies whose cells generally die when dissociated from each other unless special precautions are taken. As a consequence, they have historically been passaged and cryopreserved in LN₂ as clumps. However, there are limitations to freezing clumps of cells, as cryoprotectant penetrates them less efficiently and only a small fraction of the cells may survive after cryopreservation. Plating efficiency is typically low and clonal propagation is difficult.

To overcome the above technical issue, ID6 cells were dispersed into smaller cell aggregates prior to freezing by using a “gentle dissociation reagent” (Stem Cell Technologies) for 6 minutes and supplemented with 10 uM of ROCK inhibitor prior to freezing. Cells separated in this manner typically provided clumps of 6-8 cells, as shown in FIG. 7B.

For maintenance, ID6 piPSC were cultured on irradiated mouse embryonic fibroblasts (iMEF) feeder layers in six-well culture plates in standard hESC medium (hESCM) supplemented with 20% knockout serum replacement (KOSR, Gibco) and 4 ng/ml human FGF2. The procedure for cooling, storage, and thawing, were the same as those described in Example 4. Samples were thawed after 5 and 15 weeks of storage. Thawed cells from three samples in each treatment group were transferred to 6-well plates coated with iMEF, with cells from one vial divided equally between two wells. On day 4 after thawing and plating, five images of different areas of each culture well were captured at 40× magnification to determine colony areas relative to the control group that had been stored in LN₂. Cells were then fixed in 4% v/v paraformaldehyde in phosphate-buffered saline (PBS, Hyclone) for 2 min and stained for alkaline phosphatase activity to increase contrast. Nine images were taken at 8× magnifications to cover the entire area of the well and used to measure the total number of colonies present. All images were analyzed by the Image J software. The results are shown in FIG. 6B. As observed from Example 4 (FIG. 6A), cells cryopreserved using the medium of present invention at −80° C. (right bars) provided a similar number of colonies per well (FIG. 6B, left panel) and similar colony sizes (FIG. 6B, right panel) as storage in LN₂ (left bars), and there was no decline in cryopreservation efficiency over time (FIG. 6B). For the cells stored at −80° C. without using the medium of the present invention (center bars, FIG. 6B), significant declines in post-thaw cell survival and colony size were observed over time even after only 5 weeks, so no longer storage periods were followed. For the tests of pluripotency of the thawed cells, the same procedure described in Example 4 is followed, except that the primary antibodies didn't include NANOG. As shown in FIG. 9.B, the cells from −80° C. storage using the method of present invention also retained a pluripotent phenotype.

### Example 6. Examination of Viability and Pluripotent Features of Human iPSC Cells after Long-Term Storage at −80° C.

The medium of the present invention was also able to provide effective cryopreservation for human iPSC. The human iPSC line was derived from human umbilical cord fibroblasts reprogrammed with five factors (POU4F1, SOX2, KLF4, LIN28, and MYCL) and TP53 shRNA by using episomal plasmid transfection. Cells were cultured on Matrigel (BD Bioscience) coated six-well culture plates (Nunc) in defined mTeSR1 medium (STEMCELL Technologies). The morphology of cell colonies of human iPSC lines is similar to ID6 cells in Example 5. Therefore, before freezing, the cell colonies were also dispersed into smaller cell aggregates as described in Example 5. The procedure for cooling, storage, thawing, post-thaw viability tests, were almost the same as those described in Example 5, except that thawed cells were transferred to 6-well plates coated with Matrigel. As shown in FIG. 6C, the post-thaw survival and colonies size after 65 week of storage are almost the same for the control group (liquid nitrogen storage without using the medium of the present invention, left bars) and the treatment group (−80° C. storage using the medium of the present invention, right bars). The pluripotency tests of the thawed cells, following the similar procedure described in Example 4 except that SSEA1 was replaced by SSEA4, also demonstrated that the cryopreserved cells at −80° C. after a period of 65 weeks retained a pluripotent phenotype (See FIG. 9C).

### Example 7. Examination of Viability and Pluripotent Features of Human ESC Cells after Long-Term Storage at −80° C.

H1 hESC (WA01) were obtained from the WiCell Research Institute, Madison Wis. in 2002. The procedure for culturing, maintaining, dispersing, cooling, storage, thawing, and post-thaw viability tests, were the same as those described in Example 6. The results are shown in FIG. 6D, and the post-thaw viability after 5 and 15 week of −80° C. storage showed no decline when the medium of the present invention was used (right bar in the left panel) and were comparable with those from liquid nitrogen storage (left bar in the left panel). Without using the medium of the present invention (center bars in both channels), both cell viability and colony size were significantly lowered. As shown in FIG. 6D right panel, using the medium of the present invention caused decreased colony size, though the results were still 100% better than those stored at −80° C. without using the medium of the present invention.

Alternatively, we next used TrypLE dispersion of the H1 hESC colonies in presence of a RHO-kinase inhibitor (ROCKi, Y-27632), which protects hESC from cell death, to achieve a suspension of single cells (FIG. 7C) before mixing and freezing. We also tested the use of the medium of the present invention for liquid nitrogen storage to test whether its usage would influence the cryogenic storage efficiency. The cooling, storage, thawing procedures were the same as described above. For the post-thaw viability and functionality tests, flow cytometry was performed to provide more detailed comparison. The hESC were dispersed into single cells by TrypLE (Invitrogen) treatment for 7 min at 37° C., fixed in a Foxp3 Fixation/Permeabilization solution (eBioscience) for 1 h on ice, and incubated in 5% (v/v) donkey serum for 15 min to reduce any nonspecific binding of antibodies. Cell were then exposed to an antibody directed against POU5F1 (1:200, Santa Cruz Biotechnology) or to IgG (0.4 μg/mL; Santa Cruz Biotechnology) in the blocking buffer for 1 h. All the steps were performed in the dark on ice, and cells were washed by Permeabilization Solution (eBioscience) three times between each step. For each cell population, at least 10 000 cells were analyzed in the Accuri C6 Flow Cytometer (BD Biosciences). Data were analyzed by the FlowJo (version X) software. The results were shown in FIG. 8.

First, use of the different base media (FBS in the left panel and DMEM in the right panel) for making the medium of the present invention doesn't cause any noticeable difference in post-thaw cell survival and plating efficiency. Therefore, the medium of the present invention can be completely serum free; second, the use of the medium of the present invention did slightly improve the cryopreservation efficiency in liquid nitrogen, comparing the data demonstrated on the center left bars (using the medium of present invention for liquid nitrogen storage) and left bars (using the solely 10% DMSO for liquid nitrogen storage), without any negative effects; third, −80° C. storage using the medium of present invention (right bars) yielded almost identical cell survival and post-thaw plating efficiency as those from liquid nitrogen storage (left bars); and at last, −80° C. storage without using the medium of present invention resulted in significantly lowered post-thaw plating efficiency and viability (center right bars) than other treatments.

For the post-thaw H1 hESC cells from −80° C. storage when the medium of the present invention was used, FIG. 9D shows lineage markers expressed in embryoid bodies (EB) differentiated from cryopreserved H1 hESC: KRT7 (trophectoderm), DESMIN (mesoderm), NESTIN (ectoderm), and SOX17 (endoderm), with performed immunohistochemistry procedure similar to the above examples (characteristic biomarkers are obviously different). To achieve spontaneous differentiation of hESC via EB formation, the colonies of post-thaw hESC were dispersed by dispase/mechanical dissociation and transferred into EB differentiation medium, consisting of DMEM/F12, 15% FBS, 1% non-essential amino acids, 1 mM L-glutamine, and 0.1 mM β-mercaptoethanol, in low attachment plates (Corning). After 5 days of growth in suspension, the EB were seeded onto gelatin-coated plates and cultured in the same medium for another 9 days before fixation for immunohistochemistry.

FIG. 9E also shows cardiomyocytes differentiated from cryopreserved H1 hESC that were preserved at −80° C. using the medium of the present invention: top panel, colony of beating cardiomyocytes; lower panels, expression of cardiac marker TNNT2. Scale bar=200 μm. The post-thaw hESC were differentiated into cardiomyocytes by using the components of a kit (Cardiomyocyte Differentiation Kit; Gibco) and following the manufacturer's instructions. In brief, the hESC colonies, cultured on the Matrigel (BD Bioscience) coated plates in mTeSR1 medium (STEMCELL Technologies), were treated by Cardiomyocyte Differentiation Medium A (Gibco) for two days, followed by Cardiomyocyte Differentiation Medium B (Gibco) for another two days. The cells were then cultured in the Cardiomyocyte Maintenance Medium for a further 8 days when spontaneously contracting cardiomyocytes were appeared. The expression of cardiomyocytes marker TNNT2 was also confirmed by immunohistochemistry, procedures similar to those in the above examples.

### Example 8

Experiments using the medium of the present invention were also performed with ID6 porcine iPSC epiblast-type cells separated using different methods. Colonies (FIG. 7A) were broken into large clumps (˜100 cells) after dispase treatment and subsequent use of a cutting tool to yield uniformly sized clumps and shown in FIG. 7D. (In FIG. 7, scale bar=500 μm). Although such a method has historically been a standard method of passaging such cells, it is no longer preferred method to treat epiblast-type stem cells prior to cryopreservation. The dissociation methods discussed with respect to the foregoing Examples, and depicted in FIG. 7B and FIG. 7C, are more preferably used, and cells dissociated by such means showed positive results when stored using the medium of the present invention, as discussed above. However, to directly preserve larger colonies resulted from dispase/cutting treatment, potential treatment as preloading a certain amount of the cryoprotectant into the large colonies before mixing with the medium of the present invention are known, and may later be developed, that would be expected to yield better results when the resulting cells are stored in the medium of the present invention. The reason for that improvement lies in the fact that the presence of Ficoll in the medium of the current invention slows or delays the permeation of the cryoprotectant into the inner layer of the large colonies. Therefore, a pretreatment as loading a certain amount of the cryoprotectant alone into the large colonies prior to mixing the medium of the present invention will solve this issue. FIG. 10 confirms the cutting method without a pretreatment stated above is not the preferred method for application of the method of the present invention. FIG. 10 shows the colonies formed after mechanical dissociation of the colonies and 2 weeks of cryopreservation. FIGS. 10A and 10B depict the large colonies of FIG. 7D stored without the medium of the present invention under −80° C. and LN₂ storage conditions, respectively. FIG. 10C depicts the cells stored at −80° C. in the medium of the present invention without a pretreatment stated above.

### Example 9. Efficiency on Mid-Term Cryopreservation of Three Valuable Cell Types at −80° C.

Spermatozoa, peripheral blood mononuclear cells (PBMC), and E. coli are typical cell types that can be successfully cryopreserved in liquid nitrogen without gradual loss of cell viability during cryostorage even when storage period extends to years. However, their storage at −80° C. using homemade or commercially available cryopreservation media results in remarkable loss of viability and functionalities even after mid-term periods of storage (e.g., several months), and the rate and degree of such losses depend on cell types. Using the examples described below, we are demonstrating that those losses were prevented when cryopreservation of the invention was adopted for these cell types.

### Example 9.1. Comparison of a Medium of the Invention and Commercially Available Cryopreservation Medium for Storage of Porcine Spermatozoa at −80° C.

Improvement of the efficiency of preservation of porcine semen is critical for pig breeding improvement and is highly valuable for food industry. Cryopreservation or transportation of porcine semen in liquid nitrogen or its vapor, using liquid nitrogen dewars, dry shippers or much more expensive cryogenic freezers, is highly expensive and impractical for use in most farm operations. As a result, freshly collected semen is widely mixed with commercially available extender solutions (no cryoprotectants) at around 4° C., but that method can only maintain sperm viability for approximately one week.

To overcome that limitation, a medium of the invention was used for pig semen cryopreservation at −80° C., which enables collectors to efficiently cool and ship the sperm suspension on dry ice (available in many supermarkets) and store in regular −80° C. deep freezers. The sperm rich fraction of boar semen (˜100 ml) was collected and filtered twice through a sperm filter and placed at room temperature for 1.5 hr. Filtered semen samples (25 ml for each sample) were transferred to 50 ml conical tubes and washed by gently mixed by 1:1 (v/v) with 25 ml sperm wash medium and then centrifuged 1000×g for 7 minutes. Supernant of each sample was removed and then 5 ml of commercially available sperm extender (BF5, whose major components include egg yolk and glucose, etc.) was gently mixed with the centrifuged spermatozoa as a standard extension or suspension procedure. Each suspension sample was precooled to 4° C. in a refrigerator before freezing. For the Control the commercially available boar semen cryopreservation medium (BF5 plus 4% v/v cell culture grade glycerol), which is only valid for storage of porcine semen in liquid nitrogen, was added dropwise to the suspension with final volume ratio as 1:1, and resulted in a final volume approximately as 10 ml. The new suspension was aliquoted into 0.5 ml straws (10-20 straws) and the straws were put on dry ice for one hour and then stored in a −80° C. freezer. For the treatment groups, the BF5 was used as the base medium of the new freezing media (just as using DMEM as the base medium for stem cells in previous examples). Two treatment groups were prepared: the cryopreservation medium for Treatment A was BF5 mixed with 4% v/v cell culture grade glycerol and 20% w/v Ficoll 70; Treatment B was BF5 mixed with 4% v/v cell culture grade glycerol and 10% w/v Ficoll 70. For both treatments, the sperm suspensions in BF5 were mixed with the new Ficoll containing freezing media (A and B, respectively) with a 1:1 ratio, then aliquoted into 0.5 ml straws, put on dry ice for freezing and stored in the deep freezer (same as the Control).

After two months of storage, straws from three groups were thawed by directly plunging the straws into room temperature water. For each group, suspensions from two of the straws were diluted by 10× and cultured for 2 hrs. to evaluate post-thaw motility, and cells from other straws were collected and co-cultured with approximately 100 porcine oocytes in IVF medium for IVF efficiency evaluation. The results are listed in Table 2, below.

It is straightforward to conclude that the Treatment A, a typical embodiment of the invention, e.g. the use of the base medium with the addition of 20% w/v Ficoll 70 and 4% v/v glycerol as the permeating cryoprotectant and mixing it with cell suspension with a 1:1 ratio, significantly improved the post-thaw motility of the porcine sperm after two months of storage, whose viability and IVF efficiency is comparable to the outcome from liquid nitrogen storage. In contrast, if the medium contained either no Ficoll or an insufficient amount of Ficoll, then the post-thaw motility and functionality was severely impaired after two months of storage in a −80° C. freezer.

TABLE 2
Cryopreservation medium Treatment A, cryopreservation medium
Treatment B, and Control cryopreservation medium used for
storage of pig spermatozoa at −80° C. for two months.

| Groups | Post-thaw Motility | IVF tests |
|---|---|---|
| Control | 15% progressive | No blastocysts formed |
| (BF5 + 4% glycerol) | Very low motility | |
| Treatment A | 40% progressive | Successful blastocyst |
| (BF5 + 4% glycerol + | High motility | formation (>20%) |
| 20% Ficoll 70) | | |
| Treatment B | 20% progressive | Minimal blastocyst |
| (BF5 + 4% glycerol + | Low motility | formation (<5%) |
| 10% Ficoll 70) | | |

All the cryopreservation media were mixed with the cell suspensions with a 1:1 volume ratio.

### Example 9.2. Comparison of a Medium of the Invention and Widely Used DMSO+FBS Medium for Storage of Porcine PBMC at −80° C.

PBMCs are highly valuable for blood banking and widely used in research or biomedical applications related to immunology (including auto-immune disorders), infectious diseases, hematological malignancies, vaccine development, etc Using the combination of DMSO, FBS or BSA, and base medium (e.g. DMEM), these cells can be successfully cryopreserved in liquid nitrogen or its vapor for many years without loss of cell viabilities. However, when they are stored in −80° C. freezers, gradual cell loss has been observed, and after slightly more than one year of storage, the recovery is minimal. And using dry ice boxes for transportation of those samples previously preserved in liquid nitrogen or its vapor results in inevitable cell loss due to recrystallization during transition (warming from −120° C. or lower to −78° C. or higher). For many small clinics or hospitals, establishing cell stocks of PBMC without using expensive liquid nitrogen facilities is technically impossible. In addition, the high concentrations of FBS (generally 40% v/v) used in PBMC cryopreservation medium also significantly increase the associated cost, because the price of FBS (˜$800 to 1,000 per liter) is much higher than DMEM or other simple base medium (˜$20 per liter), and more importantly, FBS as an animal product generates contamination and regulatory issues.

To solve these practical issues, a medium of the invention was used for porcine PBMC cryopreservation in −80° C. freezers without using any FBS. Approximately 10 ml pig blood was first mixed with equal volume of PBS+2% FBS, and the cells were collected through standard density gradient centrifugation (1200 g for 10 minutes) by using the top layer. The enriched cells were washed and centrifuged again (450 g for 10 minutes) and cultured in commercially available culture medium (RPMI) supplied with diluted GM-CSF (human granulocyte-macrophage colony-stimulating factor) at 37° C. and with 5% CO₂ in an incubator for 6 days. Cells were then collected and concentrated by centrifugation and resuspended in RPMI. The new suspensions were aliquoted as 0.5 ml in each 1 ml cryovial (˜105 cells per vial). The Control group was treated with traditional cryopreservation medium, which contains 20% v/v DMSO, 40% v/v FBS and 40% v/v DMEM, by adding 0.5 ml of the medium into the cell suspension in the cryovial dropwise so that the final volume ratio between the cell suspension and cryopreservation medium is 1:1. The Treatment group was treated with the medium of invention without any FBS, which is based on DMEM with the addition of 20% v/v DMSO and 20% w/v Ficoll 70 and also mixed with the cell suspension dropwise in the cryovial with a final 1:1 volume ratio.

After mixture, the cryovials were then mounted in commercially available freezing box, Mr. Frosty, and the box was cooled in a −80° C. lab freezer overnight and the cryovials were then placed in storage boxes in the same freezer for storage. After two months of storage, the cryovials were thawed in a 37° C. water bath. The cell viability of all samples prior to freezing and post-thaw was determined using TC20™ automated cell counter. The ratios between the post-thaw viability and the viability prior to freezing for both groups are listed in Table 3, below. The results clearly demonstrated that the medium of the invention resulted in high recovery of cells, comparable to published data when PBMC are preserved in liquid nitrogen, and more importantly, efficiently prevented the cell loss during the two month storage period shown in the results of the group when traditional DMSO+ FBS medium was used. The medium of invention is also serum free (without any FBS).

TABLE 3
Cryopreservation medium of invention and standard
DMSO+ FBS medium used for storage of pig
PBMCs at −80° C. for two months.

| | Ratio of post-thaw viability |
|---|---|
| Groups | to prior-to-freezing viability |
| Control | 50.2 ± 8.5% |
| (20% v/v DMSO, 40% v/v | |
| FBS and 40% v/v DMEM) | |
| Treatment | 84.0 ± 12.7% |
| (DMEM + 20% v/v DMSO | |
| and 20% w/v Ficoll 70) | |

Both of the cryopreservation media were mixed with the cell suspensions with a 1:1 volume ratio.

### Example 9.3. Comparison Between a Medium of the Invention and Widely Used Media for Storage of E. coli Competent Cells (Typical Prokaryotic Cells) at −80° C.

E. coli competent cells are most commonly used bacterial cell types for transformation of DNA in molecular biology research and technological development. Cryopreservation of E. coli competent cells in liquid nitrogen using DMSO is a widely used protocol for long-term storage. Although many labs use high concentrations of glycerol for temporary storage in −80° C. deep freezers, the preserved cells stocks will expire after several months and using high concentration of glycerol (highly viscous) is problematic in operation.

To improve the efficiency of long-term storage of E. coli competent cells in deep freezers, a medium of the invention was tested in comparison with the treatments of using low concentration of DMSO and high concentration of glycerol when the cells were stored at −80° C. for two months. The pre-cultures of NEB® 5-alpha F'Iq competent E. coli were diluted (1:50 in LB medium at −37° C.), grown till the OD reached 0.6, and then cooled on ice. The samples were transferred to centrifuge tubes and centrifuged for 5 minutes at 3000 rpm and the pellets were then re-suspended in 25 ml DI water. This washing step was repeated three for all samples. The final pool of pellets was resuspended in 0.1M CaCl₂ water solution and aliquoted as 0.1 ml in each 0.5 ml cryovials and cooled on ice. Three cryopreservation media were prepared for three different treatments: Treatment A as 14% v/v DMSO and 0.1M CaCl₂ in DI water; Treatment B as 40% v/v glycerol and 0.1M CaCl₂ in DI water, Treatment C (a medium of the invention) as 14% v/v DMSO, 20% w/v Ficoll 70 and 0.1M CaCl₂ in DI water. For all the samples (0.1 ml cell suspension precooled on ice, as stated above) for each treatment, 0.1 ml of the corresponding cryopreservation medium was added to the cell suspension directly (i.e. volume ratio is also 1:1), and then mixing was completed by gentle shaking and samples were kept on ice for 20 mins. The cryovials were then mounted in sample storage boxes (10×5×2 cm) and directly mounted in −80° C. freezer for cooling and storage (cooling rate approximates 15-20° C./min).

After the storage period for one day, one month, and two months, respectively, cryovials from each treatment group were thawed to compare the influence of the storage periods on cell viability loss across different treatments. The value of colony forming units (CFU) for each sample was determined after performing standard culture and counting protocols for E. coli competent cells. The CFU values after one day, one and two months of storage for these treatments are listed in Table 4, below. As demonstrated in Table 4, the post-thaw colony formation efficiency for both Treatments A and B gradually decreased. In contrast, Treatment C still satisfactorily maintained colony formation efficiency, which will be beneficial for a large number of molecular biology labs to maintain their E. coli stocks in deep freezers. The CFU value resulting from Treatment A is much lower than the other two (one order of magnitude lower), because Treatment A is generally used for liquid nitrogen storage of E. coli but not for their −80° C. storage. The one day storage using Treatment C resulted in a CFU value lower than the Treatment B, but from the point of view regarding long storage periods (esp. longer than two months), its advantage is obvious. It also establishes the usefulness of the media of the invention for prokaryotic cells.

TABLE 4
Different cryopreservation media used for storage of
E. coli at −80° C. for one and two months.

| Groups (all with | Post-thaw | Post-thaw | Post-thaw |
|---|---|---|---|
| 0.1M CaCl₂) | CFU (day 1) | CFU (day 30) | CFU (day 60) |
| Treatment A | 7.4 ± 0.7 × 10⁶ | 5.7 ± 1.0 × 10⁵ | 3.2 ± 0.4 × 10⁵ |
| (DI water + | | | |
| 14% v/v DMSO) | | | |
| Treatment B | 11.5 ± 1.2 × 10⁶ | 7.3 ± 0.5 × 10⁶ | 4.2 ± 0.3 × 10⁶ |
| (DI water + | | | |
| 40% v/v glycerol) | | | |
| Treatment C | 8.4 ± 0.8 × 10⁶ | 8.2 ± 0.4 × 10⁶ | 8.5 ± 0.6 × 10⁶ |
| (DI water + | | | |
| 14% v/v DMSO + | | | |
| 20% w/v Ficoll 70) | | | |

All the cryopreservation media were mixed with the cell suspensions with a 1:1 volume ratio.

In summary of the results shown in above examples, the present invention is a simple and reliable method for long term storage of human and porcine pluripotent stem cells at −80° C., based on the use of the medium of the present invention that contains high concentration of Ficoll 70, a synthetic polymer of sucrose, which, it is believed, has not previously been used for this or comparable purposes. It is believed the success of the method is attributable to the ability of the Ficoll polymer to improve the thermal stability of the permeating cryoprotectant at non-cryogenic temperatures and prevent corresponding ice recrystallization that generally causes cell loss during long-term storage at non-cryogenic temperatures. The molecular mechanism is probably due to the physical properties of Ficoll 70, which is comprised of small spheres approximately 5 nm in radius. Slow cooling will lead to macromolecular crowding in the solution that remains unfrozen at −80° C., so that the packed Ficoll 70 spheres form a mechanical barrier that hinders enlargement of small ice crystals. Additionally, Ficoll 70 can avoid FBS in the cryopreservation solution, hence avoiding exposure of cells to animal products. From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

### BRIEF DESCRIPTION OF THE DRAWINGS

FIG. 1A. depicts the molecular dynamic demonstration of the macromolecular crowding behavior of a Ficoll-DMSO-water system at −80° C., and their interaction with a growing ice nucleus.

FIG. 1B depicts the molecular dynamic demonstration of an evenly distributed Sucrose-DMSO-water system at −80° C., and their interaction with a growing ice nucleus.

FIG. 1C depicts the molecular dynamic demonstration of an evenly distributed DMSO-water system at −80° C., and their interaction with a growing ice nucleus.

FIG. 2 depicts the molecular dynamic simulation results the values of the root-mean-square (RMS) distance of water atomic positions of three systems with the growing ice nucleus shown in FIG. 1. The top curve for DMSO-water; middle curve for sucrose-DMSO-water; and bottom curve for Ficoll-DMSO-water.

FIG. 3A depicts the SEM observation of fractured samples of the normal frozen cryopreservation solution.

FIG. 3B depicts the SEM observation of fractured samples of a medium of the present invention.

FIG. 4 depicts the optical observation of samples of (A) a frozen normal cryopreservation solution and (B) a medium of the present invention after 5 week storage at −80° C.

FIG. 5 depicts an assessment of cell recovery of naïve type O2K porcine iPSC cryopreserved with different post-mixture concentrations in the medium-cellular suspension (i.e. the concentration values are calculated after mixing embodiments of the media of the present invention with cell suspensions) of Ficoll 70 in (A) FBS based or (B) serum-free DMEM/F12 based media.

FIG. 6 depicts post-thaw recovery of colonies from the (A) naïve type O2K porcine iPSC, (B) epiblast type ID6 porcine iPSC, (C) epiblast type human iPSC and (D) epiblast type H1 hESC, over extended storage periods. For each storage period: Left bars: cells stored in liquid nitrogen. Center bars: cells stored in −80° C. freezer without using the medium of the present invention. Right bars: cells stored in −80° C. freezers using the medium of the present invention.

FIG. 7 depicts dissociation of epiblast type stem cells by different methods.

FIG. 8 depicts a comparison of efficacy of four different cryopreservation protocols performed on H1 hESC after single cell dissociation by trypsin with the aid of ROCKi. The left bars are cells stored in liquid nitrogen. For each storage period: The center left bars are for cryopreservation using the medium of the present invention in liquid nitrogen, showing that it is suitable for both −80° C. and liquid nitrogen storage or at any temperature in-between. The right center bars are cells stored in −80° C. freezer without using the medium of the present invention. The right bars are cells stored in −80° C. freezers using the medium of the present invention.

FIG. 9 depicts the expression of biomarkers characteristic of pluripotency of all above four stem cell types (in the same order as in FIG. 6) after recovery from cryopreservation using the medium of the present invention at −80° C.

FIG. 10 depicts the morphologies of ID6 porcine iPSC colonies, which were broken into large clumps (˜100 cells), following 2 weeks of cryopreservation.

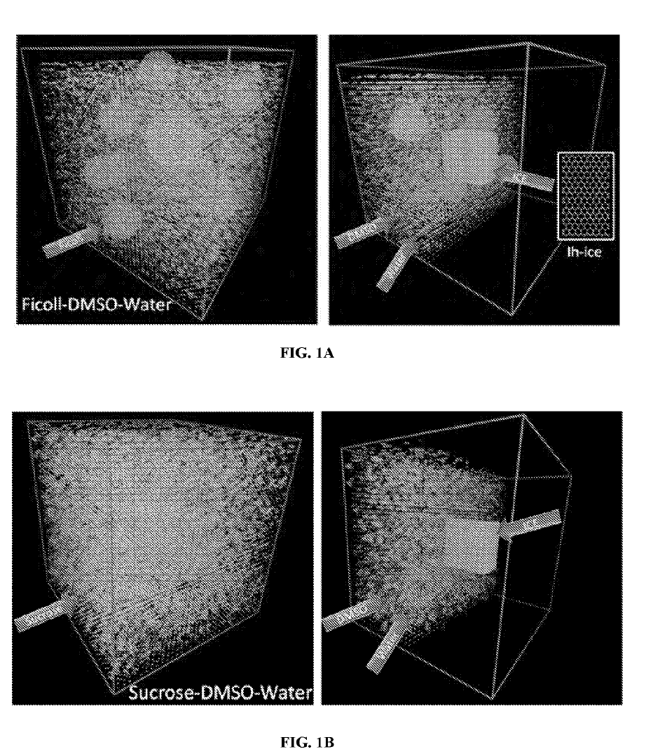

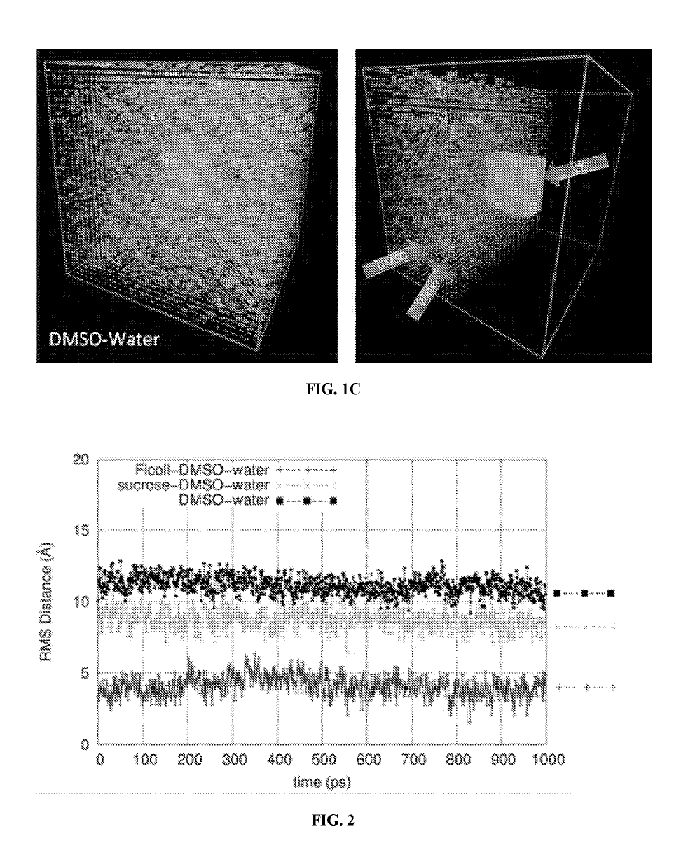

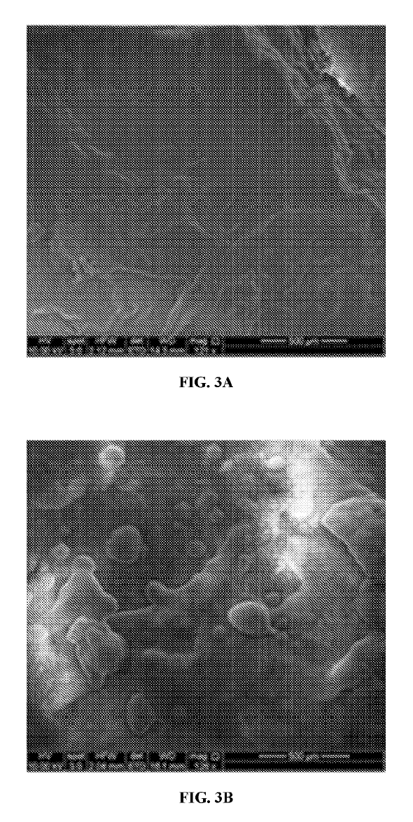

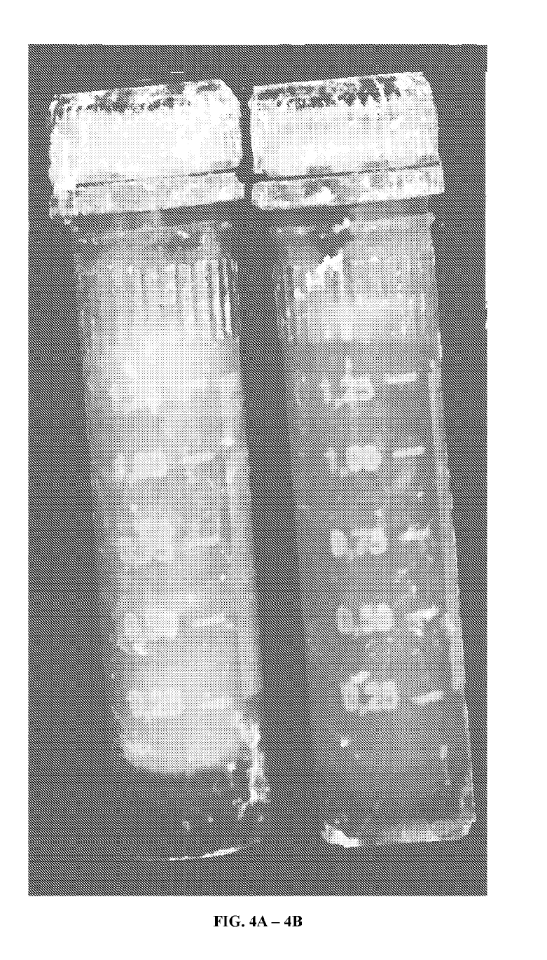

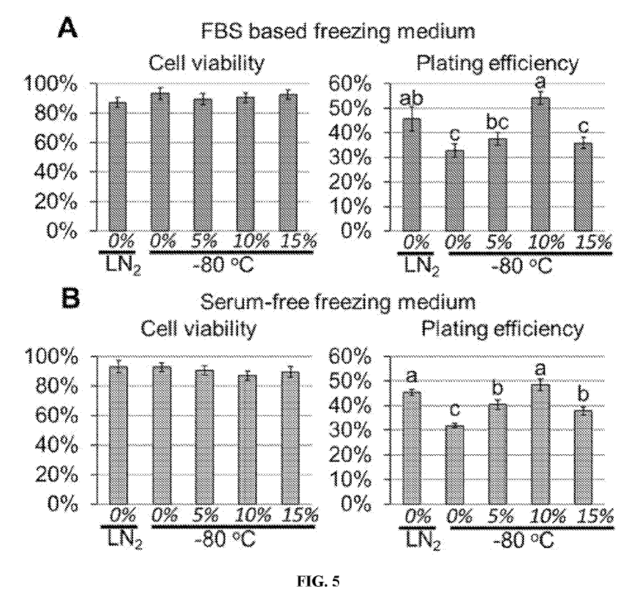

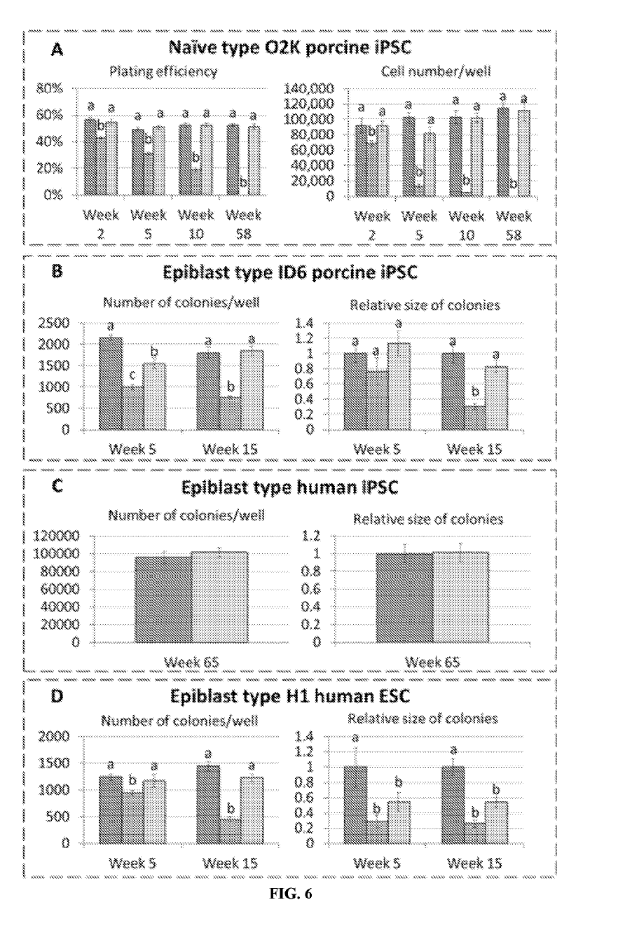

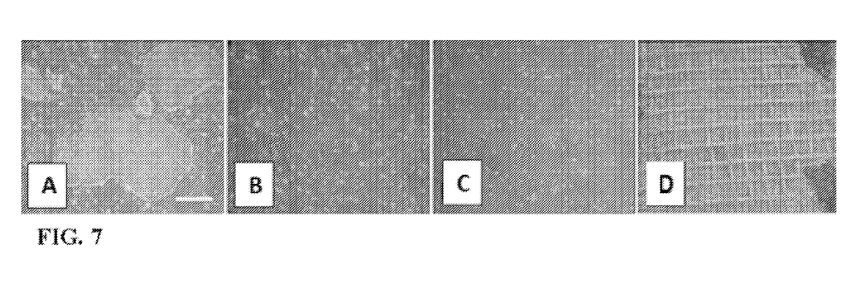

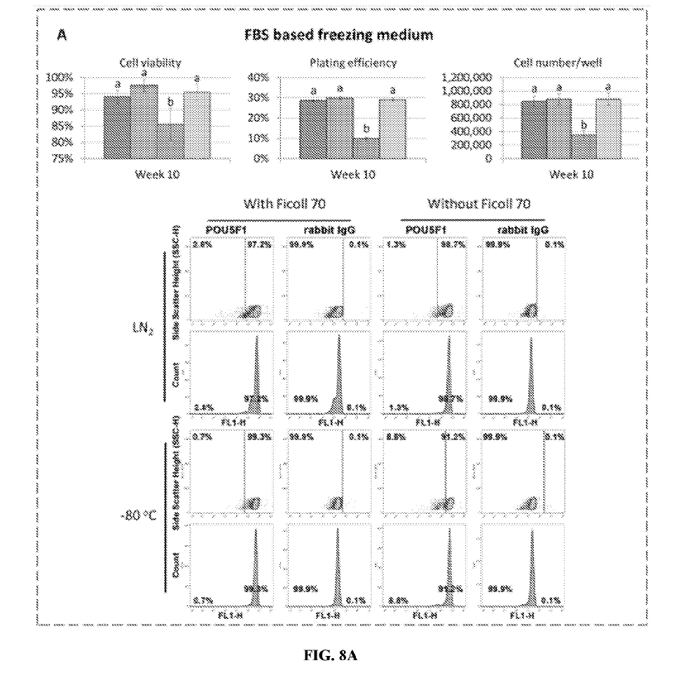

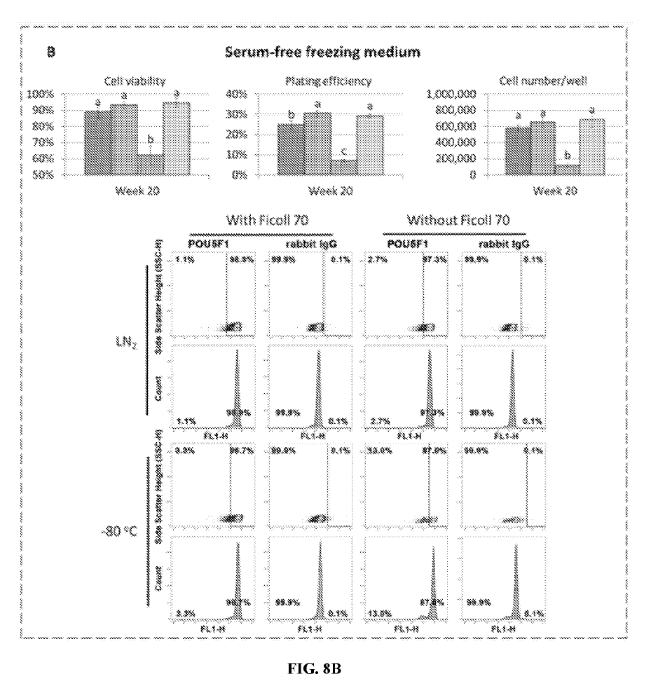

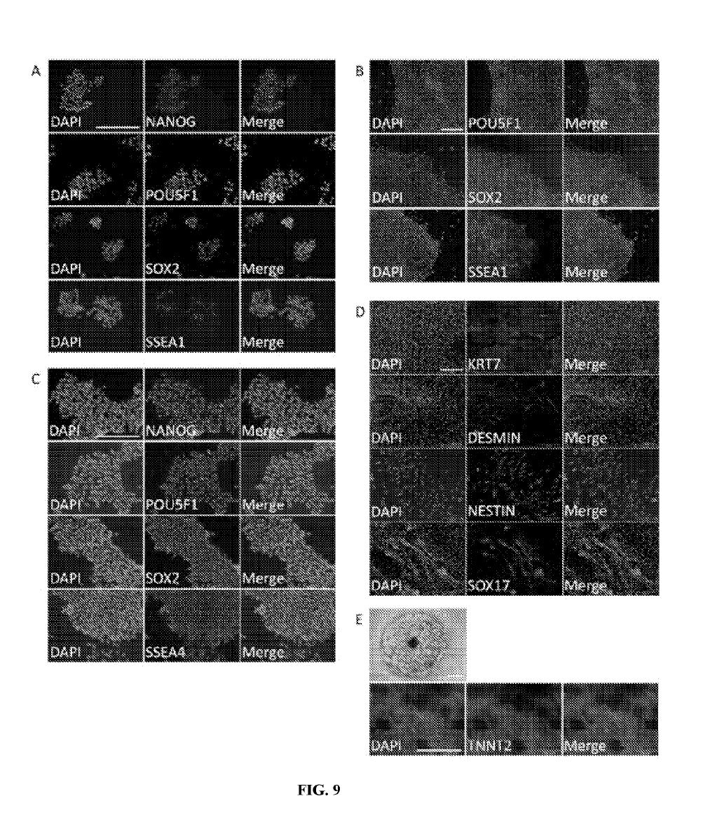

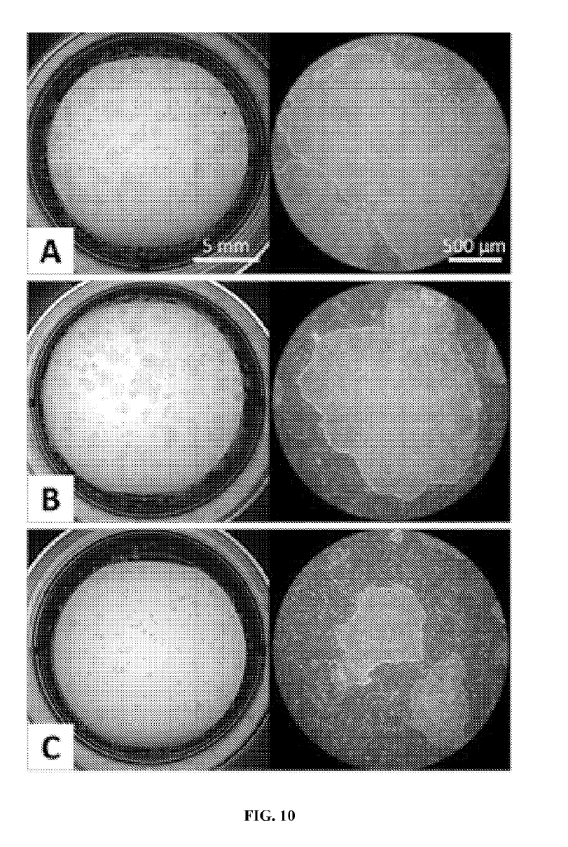

### REFERENCES

Mazur, P. Cryobiology: the freezing of biological systems. Science 168, 939-949 (1970).
Mazur, P. Freezing of living cells: mechanisms and implications. Am J Physiol. 247, 125-142 (1984).
Valeri, C. R. & Pivacek, L. E. Effects of the temperature, the duration of frozen storage, and the freezing container on in vitro measurements in human peripheral blood mononuclear cells. Transfusion 36, 303-308 (1996).
Cohen, R. I., Thompson, M. L., Schryver, B., Ehrhardt, R. O. Standardized cryopreservation of pluripotent stem cells. Curr Protoc Stem Cell Biol. Unit 1C.14 (2014).
Massie, I., Selden, C., Hodgson, H., Fuller, B. Storage temperatures for cold-chain delivery in cell therapy: a study of alginate-encapsulated liver cell spheroids stored at −80° C. or −170° C. for up to 1 year. Tissue Eng Part C Methods. 19:189-95 (2013)
Fahy, G. M. & Wowk, B. Principles of cryopreservation by vitrification. Methods Mol Biol. 1257, 21-82 (2015).
Jin, B., Mochida, K., Ogura, A., Koshimoto, C., Matsukawa, K., Kasai, M., Edashige, K. Equilibrium vitrification of mouse embryos at various developmental stages. Mol Reprod 79, 785-94 (2012).
Baudot, A. & Odagescu, V. Thermal properties of ethylene glycol aqueous solutions. Cryobiology 48, 283-94 (2004).
Forsyth, M. & MacFarlane, D. R. Recrystallization revisited. Cryo-Letters 7, 367-378 (1986).
Gao, D. & Critser, J. K. Mechanisms of cryoinjury in living cells. ILAR J 41, 187-196 (2000).
Venketesh, S. & Dayananda, C. Properties, potentials, and prospects of antifreeze proteins. Crit Rev Biotechnol. 28, 57-82 (2008).
Chantelle, J. et al. Small molecule ice recrystallization inhibitors enable freezing of human red blood cells with reduced glycerol concentrations. Sci Rep 5, 9692 (2005).
Dauty, E., & Verkman, A. S. Molecular crowding reduces to a similar extent the diffusion of small solutes and macromolecules: measurement by fluorescence correlation spectroscopy. J. Mol. Recognit 17, 441-447 (2004).
Ellis, R. J. Macromolecular crowding: obvious but underappreciated. Trends Biochem Sci. 26, 597-604 (2001).
Amit, M. et al. Clonally derived human embryonic stem cell lines maintain pluripotency and proliferative potential for prolonged periods of culture. Developmental biology 227, 271-278 (2000).
Ezashi, T., Das, P. & Roberts, R. M. Low O2 tensions and the prevention of differentiation of hES cells. Proceedings of the National Academy of Sciences of the United States of America 102, 4783-4788 (2005).
Roberts, R. M., Yuan, Y., Genovese, N. & Ezashi, T. Livestock Models for Exploiting the Promise of Pluripotent Stem Cells. ILAR Journal 00 (2015).
Ezashi, T., Yuan, Y. & Roberts, R. M. Pluripotent Stem Cells from Domesticated Mammals. Annual review of animal biosciences (2015).
Telugu, B. P. et al. Leukemia inhibitory factor (LIF)-dependent, pluripotent stem cells established from inner cell mass of porcine embryos. The Journal of biological chemistry 286, 28948-28953 (2011)
Lee, J. E. & Lee, D. R. Human Embryonic Stem Cells: Derivation, Maintenance and Cryopreservation. International journal of stem cells 4, 9-17 (2011).
Fujioka, T., Yasuchika, K., Nakamura, Y., Nakatsuji, N. & Suemori, H. A simple and efficient cryopreservation method for primate embryonic stem cells. The International journal of developmental biology 48, 1149-1154 (2004).
Reubinoff, B. E., Pera, M. F., Vajta, G. & Trounson, A. O. Effective cryopreservation of human embryonic stem cells by the open pulled straw vitrification method. Human reproduction 16, 2187-2194 (2001).
Richards, M., Fong, C. Y., Tan, S., Chan, W. K. & Bongso, A. An efficient and safe xeno-free cryopreservation method for the storage of human embryonic stem cells. Stem cells 22, 779-789 (2004).
Ezashi, T. et al. Derivation of induced pluripotent stem cells from pig somatic cells. Proceedings of the National Academy of Sciences of the United States of America 106, 10993-10998 (2009).
McElroy, S. L. & Reijo Pera, R. A. Culturing human embryonic stem cells in feeder-free conditions. CSH protocols 2008, pdb prot5044 (2008).
Martin-Ibanez, R. et al. Novel cryopreservation method for dissociated human embryonic stem cells in the presence of a ROCK inhibitor. Human reproduction 23, 2744-2754 (2008).
Kurosawa, H. Application of Rho-associated protein kinase (ROCK) inhibitor to human pluripotent stem cells. Journal of bioscience and bioengineering 114, 577-581 (2012).
Watanabe, K. et al. A ROCK inhibitor permits survival of dissociated human embryonic stem cells. Nature biotechnology 25, 681-686 (2007).
Baharvand, H., Salekdeh, G. H., Taei, A. & Mollamohammadi, S. An efficient and easy-to-use cryopreservation protocol for human ES and iPS cells. Nature protocols 5, 588-594 (2010).
Okita, K. et al. A more efficient method to generate integration-free human iPS cells. Nature methods 8, 409-412 (2011).
Lee, K. et al. Engraftment of human iPS cells and allogeneic porcine cells into pigs with inactivated RAG2 and accompanying severe combined immunodeficiency. Proceedings of the National Academy of Sciences of the United States of America 111, 7260-7265 (2014).
Girish, V. & Vijayalakshmi, A. Affordable image analysis using NIH Image/ImageJ. Indian journal of cancer 41, 47 (2004).
Johnson, L. A., Weitze, K. F., Fiser, P., Maxwell, W. M. C., Storage of Boar Semen, Animal Reproduction Science 62, 143-172 (2000).
Yang, Jun, et al. The effects of storage temperature on PBMC gene expression, BMC Immunology 17, 6-21 (2016).
Cody, William L., et al. Skim Milk Enhances the Preservation of Thawed −80° C. Bacterial Stocks, J Microbiol Methods 75, 135-138 (2008).

1. A medium for preserving cells at a non-cryogenic freezing temperature comprising:
a hydrophilic and nontoxic macromolecule;
an aqueous liquid; and
a cryoprotectant;
wherein the macromolecule is at a concentration in the medium equal to or greater than about 20% (w/v), and
wherein molecules of the macromolecule form compact three-dimensional structures that are spherical in shape when dissolved in the aqueous liquid;
wherein when the medium is in use and is at the non-cryogenic freezing temperature, the compact and spherical structures are concentrated in an unfrozen portion of the medium with the cells being preserved; and
wherein a crowding effect prevents ice recrystallization during storage at the non-cryogenic temperatures.

2. (canceled)

3. (canceled)

4. The medium of claim 1, wherein the concentration of the macromolecule in the medium is about 25% (w/v) or greater.

5. The medium of claim 4, wherein the concentration of the macromolecule in the medium is about 35% (w/v) or greater.

6. The medium of claim 5, wherein the concentration of the macromolecule in the medium is about 50% (w/v) or greater.

7. The medium of claim 1, wherein the cryoprotectant is at a concentration equal to or greater than about 20% of the concentration of the macromolecule in the medium.

8. The medium of claim 7, wherein the concentration of the cryoprotectant in the medium is equal to or greater than about 75% of the concentration of the macromolecule in the medium.

9. The medium of claim 8, wherein the concentration of the cryoprotectant in the medium is equal to or greater than about 100% of the concentration of the macromolecule in the medium.

10. The medium of claim 1, wherein the macromolecule is a polymer.

11. The medium of claim 10, wherein the polymer comprises molecules that form the compact three-dimensional structures that are approximately spherical in shape when dissolved in the aqueous liquid.

12. The medium of claim 11, wherein the polymer is selected from the group consisting of spherical hydrophilic polysaccharides, polymerized cyclodextrin or saccharides, globular proteins or spheroproteins, spherical glycoproteins formed by attaching oligosaccharide chains to those globular proteins, other derivatives of those globular proteins and combinations thereof.

13. The medium of claim 12, wherein the polymer is a hydrophilic polysaccharide.

14. The medium of claim 13, wherein the polymer is a polymer formed by the copolymerization of sucrose and epichlorohydrin.

15. (canceled)

16. (canceled)

17. (canceled)

18. The medium of claim 1, wherein the cells are eukaryotic cells.

19. The medium of claim 18, wherein the suspended cells are mammalian cells and the mammalian cells are selected from the group consisting of murine cells, porcine cells, human cells, and combinations thereof.

20. The medium of claim 18, wherein the mammalian cells are selected from the group consisting of stem cells, somatic cells, reproduction cells and combinations thereof.

21. The medium of claim 1, wherein the cells are prokaryotic cells.

22. The medium of claim 1, wherein the compact approximately spherical structures are about 100 nm (nanometer) or less in their widest dimension.

23. The medium of claim 17, wherein the compact approximately spherical structures comprise structures ranging from about 1 to 50 nm in their widest dimension.

24. (canceled)

25. The medium of claim 1, wherein the medium is substantially free of serum, animal proteins or human proteins.

26. A method for preserving cells at a non-cryogenic freezing temperature comprising:
providing a cryopreservation medium comprising a hydrophilic and nontoxic macromolecule, a cryoprotectant, and an aqueous liquid, wherein the macromolecule is at a concentration in the medium greater than about 10% (w/v), and wherein the macromolecule forms a highly compact approximately spherical structure when dissolved in the aqueous liquid;
using the medium to suspend the cells to form a medium-cellular suspension;
cooling the medium-cellular suspension to the non-cryogenic freezing temperature, wherein the non-cryogenic freezing temperature is about −85° C. or higher; and
maintaining the medium-cellular suspension at or near the non-cryogenic freezing temperature or a different non-cryogenic freezing temperature, without losing water due to sublimation or without any need for lyophilization or freeze drying processing, for a time period longer than three weeks while maintaining post-thaw cell survival rates of the cells equal to or about the same as would be obtained for storage of the cells in liquid nitrogen for the period of time.

27.-44. (canceled)
